(19) United States
(12) Patent Application Publication
(10) Pub. No.: US 2008/0215099 A1
(43) Pub. Date: Sep. 4, 2008

(54) Orthodontic bone screw

(76) Inventors: Alan R. Balfour, Petaluma, CA (US); William C. Machata, Kohler, WI (US); Joseph M. Caruso, Redlands, CA (US); Joseph Edward Carchidi, West Bridgewater, MA (US)

Correspondence Address: John A. Haug, P.O. Box 386, West Harwich, MA, 02671, US

(21) Appl. No.: 12/002,421

(22) Filed: Dec. 17, 2007

Related U.S. Application Data
(62) Division of application No. 11/287,673, filed on Nov. 28, 2005, now abandoned.

(51) Int. Cl. A61B 17/56; A61C 8/00

(52) U.S. Cl. 606/306; 433/174; 606/301; 606/305

(57) ABSTRACT

A mechanism for separating the driver tip (28) of a driver tool (27) from an orthodontic bone screw (25) is shown. The mechanism includes a driver sleeve (36) formed with a female thread (38) that is threaded onto a male threaded portion (39) of the driver tool adjacent to the driver tip with the sleeve adapted to engage and push-off the head of the bone screw when rotated sufficiently.

### CROSS REFERENCE TO RELATED APPLICATIONS

This is a Divisional of U.S. Ser. No. 11/287,673, filed Nov. 28, 2005.

### FIELD OF THE INVENTION

This invention relates generally to orthodontic treatments and more particularly to a bone screw for use in intra-oral orthodontic corrections.

### BACKGROUND OF THE INVENTION

Presently, a variety of bone screws are commercially available for use for orthodontic anchorage; however, many of these screws generally are intended for retaining tissue grafts and bone plates and to assist in the purpose of craniomaxillofacial reconstruction rather than for use as an orthodontic fixation post. Since such screw geometries were not intended for orthodontic use, they have several limitations relative to such use.

Many bone screws that are available at the present time require one to first perform a soft tissue dissection in the area that the screw will be inserted, then to drill a pilot hole at the desired location for screw insertion. This multiple step surgical procedure for preparation to insert the screws is not only cumbersome, but also it requires skills that are not usually performed by an orthodontist. In addition, by following this procedure, the orthodontist must relocate the predrilled pilot hole in order to insert the self-threading bone screw. Since most of these surgical procedures require small diameter bone screws, relocating the pilot hole prior to insertion of the bone screw can be difficult. Once inserted, these defined screws are typically under an immediate and continuous load incident to the use as an orthodontic anchor, which typically is not a design criterion of the passive retentive maxillofacial bone screw. In general, these maxillofacial screws were developed for the purpose of lag screw retention to hold multiple bone segments together in a passive loading condition, rather than that as a stand alone screw to be used for immediate orthodontic anchorage and multi-axis loading. Yet another limitation is that many maxillofacial reconstructive screws do not incorporate a desired fixation post head for orthodontic appliance attachment; in general these screws are used sub-gingivally and require a low profile head in order to prevent soft tissue irritation.

### SUMMARY OF THE INVENTION

An object of the present invention is to provide an improved intra-oral craniomaxillofacial rigid fixation screw post for primary anchorage of orthodontic appliances. Another object of the invention is the provision of an orthodontic screw that overcomes the limitations noted above of using maxillofacial reconstruction bone screws for the purpose of orthodontic post anchorage and immediate multi-axis post loading. Yet another object of the invention is the provision of an orthodontic bone screw that can easily pierce and penetrate through the soft tissue and directly into the host bone without the surgical requirements for soft tissue dissection or pre-drilling of a pilot hole. Still another object of the invention is the provision of an orthodontic screw that will enable an orthodontist to insert the screw with the sell-piercing and penetrating tip and self tapping locking threads in a single minimally invasive surgical operation. Another object of the invention is the provision of an orthodontic bone screw that once it is rigidly inserted into the host bone, standard orthodontic appliances can be attached to the exposed driving head of the screw. Another object of the invention is the provision of an orthodontic bone screw that does not permanently integrate with the host bone thereby allowing for removal at the completion of orthodontic treatment.

These and other objects of the invention will be apparent from the following description taken with reference to the accompanying drawings.

Briefly stated, an orthodontic, skeletal anchorage bone screw made in accordance with a preferred embodiment of the invention incorporates a sharp tissue piercing, pin-point, tack tip in series with an immediate loading, self-locking, double tapered fixation thread. Distal to the tip and body of the bone screw is a generally cylindrical, dome shaped head that incorporates features for the attachment of standard orthodontic appliances. In use, an orthodontist can easily attach the anchorage bone screw head onto a spline driver tool and insert the screw into the host bone in a single operation. Since the screw is designed for easy insertion without the requirements for soft tissue dissection or a pre-drilled pilot hole preparation, it is particularly beneficial for an orthodontist who may not be trained in dissection procedures. If desired, the screw thread, such as the uppermost portion thereof, could include suitable surface texturing to enhance functional stability. After the double, self-locking, tapered thread is rigidly fixated into the bone with the one-step surgical procedure, immediate attachment of a standard orthodontic appliance can occur. The invention allows an orthodontist to take advantage of the intra-oral anchorage post for orthodontic movement of the surrounding teeth. Further, the screw provides an orthodontist with a cost effective, complete patient compliant, functional alternative to extra oral orthodontic procedures.

### BRIEF DESCRIPTION OF THE DRAWINGS

For a more complete understanding of the present invention and further advantages thereof, reference is now made to the following detailed description of the preferred embodiment taken in conjunction with the drawings in which:

FIG. 1 is an elevational view of a self-positioning and self-starting orthodontic post fixation bone screw made in accordance with the preferred embodiment of the invention;

FIG. 1(a) is an elevational view of a modification of the FIG. 1 bone screw;

FIG. 2 is a top plan view of the FIG. 1 structure;

FIG. 3 is an elevational view of a delivery and driver tool for the FIG. 1 screw;

FIG. 4 is similar to FIG. 3 but shows a detachable insert driver tip 28 and an oversized driver handle 29 separated from each other;

FIG. 5 is a broken away, enlarged tip portion of driver tip 28 of FIGS. 3 and 4; and

FIG. 6 is a top plan view of the FIG. 5 tip 28.

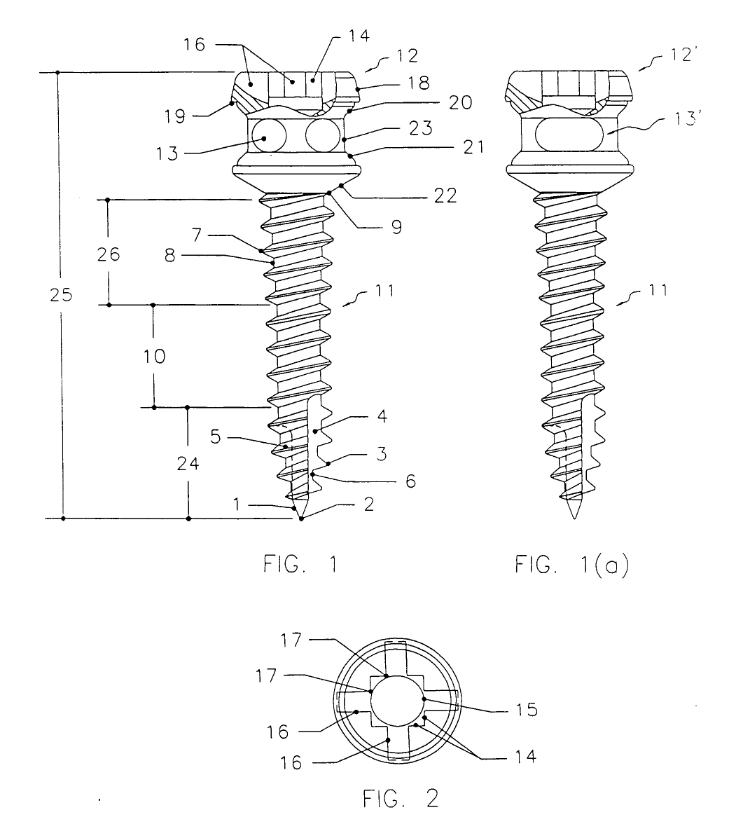

### DETAILED DESCRIPTION OF THE PREFERRED EMBODIMENTS

References made in the specification and claims to a particular orientation, such as upper, bottom and the like are made with respect to the orientation as shown in the drawings.

As shown in FIG. 1, the self-positioning and self-starting orthodontic bone screw of the preferred embodiment of the invention comprises an apical screw end 1 having a sharp tissue piercing pin-point tack tip 2 and an immediate loading, self-locking, double tapered threaded body 11. Distal to apical end 1 and threaded body 11 is a cylindrical, generally domed shaped driving head 12 that incorporates attachment features for orthodontic appliances. The pin-point tack tip 2 is used to locate, pierce and penetrate through the soft tissue as well as maintain a positional location in the host bone preventing the need for soft tissue dissection. Once pin-point tack tip 2 locates and tacks onto the host bone, the screw is rotated clockwise to immediately engage the apical cutting flutes 4 and 5 of the gradually increasing diameter bottom tapered fixation thread 24. As designed, the cutting flutes 4 and 5, which are located rotationally 180 degrees apart from each other, vary in length to assist in the self-tapping of the screw without jeopardizing the structural characteristics of the thread.

Going from the apical end of threaded body 11, a first axial length portion 24 comprises a tapered thread having increasing minor 6 and major 3 thread diameters followed by a second axial length portion 10 of a constant (minor and major) thread diameter. The length of second axial length portion 10 is varied in dependence on the overall length of bone screw 25. A third axial length portion 26 of a tapered (increasing minor 8 and major 7) thread diameter is formed adjacent to the second axial length portion 10. The tapered thread of axial length portion 26, with its increasing thread diameter, rigidly locks into the bone as the screw is driven in to seating neck 22, to be discussed.

By means of the double, self-locking emergence tapered thread, the screw can be easily inserted while maximizing crestal bone fixation. The coronal portion of the upper tapered thread geometry 26 finishes at the major diameter thread dimension 9 flush with the apical portion of seating neck 22, to be discussed, without any minor diameter thread relief geometry, to enhance the torsional strength characteristics of the screw. The screw, with its unique pin-point 2 and double, self-locking, tapered thread geometry 11, allows an orthodontist to easily and rigidly fixate the screw into a surgical site in a one-step procedure without the requirement for any soft tissue dissection. If desired, the screw thread, particularly the third axial length portion 26, can include suitable surface texturing to enhance functional stability.

The coronal end of the self-positioning and sell-starting orthodontic bone screw 25 incorporates a generally cylindrical dome shaped driving head 12 that acts as an anchorage post for the attachment of standard orthodontic appliances. Central to the dome shaped head 12 are two symmetrically located radially extending cross-holes 13 that can be utilized for orthodontic wire attachment and multi-axis loading. In addition, this central area 23 incorporates symmetrical undercut grooves 20, 21, respectively, forming a circumferentially extending annular recess that can be used for orthodontic band attachment. Finally, this grooved area 23 has an upper snap attachment configuration 19 comprising a generally right angle circumferential shoulder formed in the upper surface adjacent to groove 20 that allows for the attachment and use of a variety of orthodontic appliances to provide tooth movement.

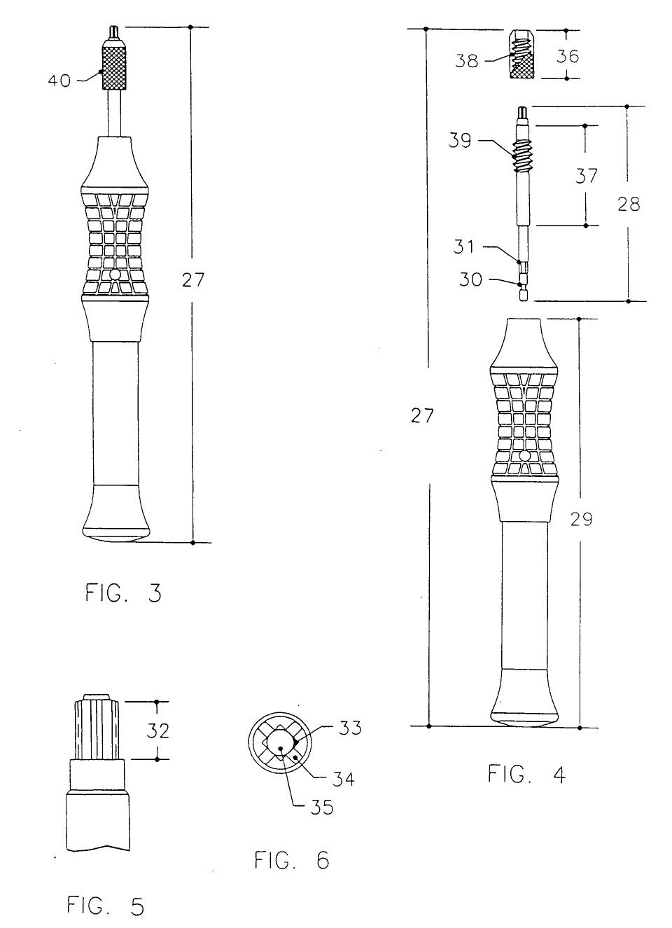

On the bottom side of the bone screw head 12 is a beveled seating neck 22 that acts as a seating surface when the screw is placed perpendicular to the surface of the bone or off-axis to some pre-determined angle by an orthodontist. Opposite beveled seating neck 22 is a tissue compatible, smooth, cylindrical dome shaped surface 18. In the center of dome shaped surface 18 is a combined square recess 14 and cross-slots 16, providing a spline 17. This combined square and cross-slot spline is used to drive bone screw 25 in or out of the surgical site using a corresponding square block and cross-rib spline pick-up and driver tool 27. Distal to the internal features used for driving the screw is a recessed bored hole 15 or cylindrical bored surface used to pick up and deliver bone screw 25 to the surgical site using the corresponding tapered cylindrical tip spline pick-up and driver tool of FIGS. 3-6.

As noted above, to deliver orthodontic screw 25 to the host bone site, FIGS. 3-6 show a press-fit pick-up and delivery spline driver tool 27. Driver tool 27 comprises a detachable insert driver tip 28 and an oversized driver handle 29. Detachable insert driver tip 28 is connected and driven by oversized handle 29 and has an insert ring 30 and centrally disposed milled male hexagonal feature 31. Disposed at the opposite end of driver tip 28 from the connecting and driving features 30, 31, is a bone screw spline driver head 32, see, FIGS. 5 and 6. Male spline driver head 32 comprises a combined male square block 33 and cross-bar 34. Distal to spline driver head 32 is a tapered cylindrical shaft tip 35 of increasing diameter as one goes in a direction away from the free end thereof that friction locks into the corresponding recessed bore 15 in bone screw 25 so that it can be easily picked up and delivered by the dentist or orthodontist to the surgical site to prevent any risk of contamination.

Once bone screw 25 is inserted into and seated in the host bone, driver tip 28 can be easily detached from dome shaped driving head 12 of the bone screw by using a push-off driver sleeve 36. This is achieved by turning knurled surface 40 of push-off driver sleeve 36 on shaft 37 in a clockwise direction to axially slide driver sleeve 36 downward until it hits and pushes off of driver head 12 of the bone screw. The axial downward push-off force, which pushes off bone screw head 12, is generated by translating the rotational female thread 38 force on the driver sleeve 36 to the mating male thread 39 on driver shaft 37. Spline drive head 32 can also be incorporated into a contra-angle driver (not shown) to allow for electrical driver insertion of screw 25.

Although the invention has been described with regards to a specific preferred embodiment thereof, variations and modifications will become apparent to those of ordinary skill in the art. It is therefore the intent that the appended claims be interpreted as broadly as possible in view of the prior art as to include all such variations and modifications.

1-9. (canceled)

10. A driver tool for use with an orthodontic bone screw having a generally cylindrical driving head, the head having a coronal end surface formed with recessed driving features including a polygonal recess, as seen in end view, and diametrically extending cross grooves are formed in the coronal end surface of the driving head and a cylindrical bored surface extending centrally and axially from the polygonal recess, the driver tool comprising a tip end formed with a block shaped as a polygon in end view having side walls, ribs extending from selected side walls of the block and a centrally located cylindrical tapered projection extending axially beyond the block, said block, ribs and projection sized to fit in the respective polygonal recess, cross grooves and cylindrical bored surface and a mechanism to separate the driver tool from a bone screw to which it has been engaged for mounting in a host bone.

11. (canceled)

12. A driver tool according to claim 10 in which the tip end is formed on a shaft and the mechanism includes a threaded portion formed on the shaft adjacent to the tip end and a push-off driver sleeve having an internal thread is threadingly engaged to the thread on the threaded portion of the shaft, the push-off driver sleeve, upon rotation thereof, being engageable with the head of a bone screw to which the tip end is engaged, the push-off driver sleeve, upon further rotation adapted to exert a force against the head of the bone screw to move the tip end out of engagement with the bone screw.

13. A driver end surface of the tool for use with an orthodontic bone screw having a generally cylindrical driving head, the head having a coronal end surface formed with recessed driving features, comprising a driver tool having a tip end with a configuration shaped for receipt in and engagement with the said recessed driving features and a mechanism to separate the driver tool from a bone screw to which it has been engaged for mounting in a host bone.

14. A driver tool according to claim 13 in which the tip end is formed on a shaft and the mechanism includes a threaded portion formed on the shaft adjacent to the tip end and a push-off driver sleeve having an internal thread is threadingly engaged to the thread on the threaded portion of the shaft, the push-off driver sleeve, upon rotation thereof, being engageable with the head of a bone screw to which the tip end is engaged, the push-off driver sleeve, upon further rotation adapted to exert a force against the head of the bone screw to move the tip end out of engagement with the bone screw.
